(19) United States
(12) Patent Application Publication
(10) Pub. No.: US 2023/0346493 A1
(43) Pub. Date: Nov. 2, 2023

(54) AUTOMATED SURGICAL SUPPORT SYSTEM FOR EYE SURGERY

(71) Applicant: Alcon Inc., Fribourg (CH)

(72) Inventor: Paul R. Hallen, Colleyville, TX (US)

(21) Appl. No.: 18/303,190

(22) Filed: Apr. 19, 2023

Related U.S. Application Data
(60) Provisional application No. 63/363,744, filed on Apr. 28, 2022.

(51) Int. Cl. A61B 34/32; A61B 90/50; A61F 9/007

(52) U.S. Cl. CPC A61B 34/32; A61B 90/50; A61F 9/007

(57) ABSTRACT

A surgical support system with a pivot mechanism for stable relocating of a surgical tool interface with an eye of a patient. The system includes hardware with a surgical tool securing device. The hardware and system place an intentional limited range of motion and stability on the surgical tool during the procedure. However, a pivot mechanism is incorporated into hardware of the system to facilitate the stable transfer of surgical interface from one of a temporal side preplaced cannula to a nasal side preplaced cannula. Thus, inefficient reconfiguring of the system or the patient between different eye side surgical procedures may be avoided. Further, the techniques employed may also be applicable during transfer between surgical procedures at different eyes of a patient.

### BACKGROUND

Over the years, many dramatic advancements in the field of eye surgery have taken place. In various procedures, a few different types of tools are generally employed. For example, an interventional tool that is tasked with directly engaging with and affecting a part of the eye may be utilized. A common example of such a tool is a vitrectomy probe utilized in a vitrectomy. A vitrectomy is the removal of some or all of the vitreous humor from a patient's eye. In some cases, where the surgery was limited to removal of clouded vitreous humor, the vitrectomy may constitute the majority of the procedure. However, a vitrectomy may accompany cataract surgery, surgery to repair a retina, to address a macular pucker or a host of other issues.

Of course, a variety of other instruments may be employed in addition to or apart from a vitrectomy probe. For example, following some degree of a vitrectomy application, forceps or scissors may be utilized to address an eye condition. This may be followed by the introduction of yet another instrument or perhaps the re-introduction of the vitrectomy probe in continuing the surgical procedure.

Regardless of the particular instrument, hazards associated with the insertion of an instrument into a patient's eye are multiplied. Each insertion risks leakage and the surgeon's own eyes need to adjust and re-adjust to the proper positioning of the tool to avoid injury. The surgeon's steadiness of instrument delivery must be repeated. This challenge may be increased given the ever-decreasing instrument size. For example, probe or instrument needles that traditionally may have been about 23 gauge may now be about 25 or 27 gauge. This translates to reducing a needle diameter from just under about 0.5 mm to less than about 0.4 mm. Once more, the dimensions of the eye are quite small. That is, the distance from the entry point of the instrument at the front of the eye to the delicate retina features at the back of the eye will generally be less than a couple of inches.

Given that the surgeon is working with a surgical implement of minimal dimensions in the small space of the eye; automated, robot assisting systems may be useful to aid the surgeon in such procedures. For example, the patient may be placed in a flat or horizontal orientation on an operating bed with the patient's head stably positioned facing upward. A robotic assistance unit may be utilized for holding surgical instruments above the patient's eye. Instead of the surgeon directly holding the desired instrument, the system may include a tool holder or securing device which directly controls the position of the instrument. The surgeon, in turn, may manipulate an implement of the system which is used to guide movements of the instrument.

Utilizing an indirect implement to manipulate the stabilized instrument instead of directly holding the instrument offers certain safety advantages. Notably, precision of instrument positioning during the surgery may be vastly improved. For example, scaling may be utilized wherein a degree of movement by the surgeon with the guiding implement is scaled down. Often the scaling is at a 1:7 ratio with the instrument moving 1/7^{th} the degree that the guiding implement is moved by the surgeon. In other words, for every inch that the guiding implement is moved by the surgeon, 1/7^{th} of an inch of movement will actually take place by the instrument in the patient's eye. Of course, this is only exemplary and any number of practical differing degrees of scaling may be utilized.

As such robotically assisted procedures take place, the surgeon may also be viewing the procedure indirectly at a monitor that presents an enlarged and visually enhanced view of the interior of the eye during the procedure. Thus, another degree of added safety and precision may be provided.

Yet another degree of safety may be provided by such robotic systems where the degree of flexibility of movement is intentionally limited. That is, rather than allow the tool holder to take on a variety of different angular orientations, the tool holder may be limited to a single axial orientation relative to the eye. For example, the instrument may be directed to enter the eye through a preplaced cannula at a 45° offset angular orientation at the sclera of the eye. This offset location may be to the nasal side of the eye or the temporal side of the eye. Regardless, the system may intentionally limit the instrument entry to this matching angular mode of entry. In this way, the instrument is limited to a single axis of advancement or withdrawal so as to avoid any accidental, unintended movement of the instrument along any other axis when inside the eye.

While the sum total of these robotically assisted limitations does help avoid unintended eye injury, the limitations also introduce an element of significant inefficiency. Namely, once a procedure is run through one preplaced cannula of a given orientation, the need to enter through another location may remain. For example, following an instrument procedure through a nasal side cannula, a subsequent procedure through a temporal side cannula may be sought. However, due to the safeguarding system limitations, the ability to simply reorient the system to take on a new and different angular mode of entry may be difficult. For example, reorienting the entire patient and operating table for sake of attaining a new entry angle by the same system may be performed. However, this may lead to delay and introduce new hazards to an already delicate surgical undertaking. As a result, surgeons are often left with the undesirable option of choosing between a less safe, unassisted procedure or one in which cumbersome delays and new, potentially hazardous inefficiencies may be introduced.

### SUMMARY

An automated surgical support system to facilitate eye surgery is described. The system includes a tool securing device that is supported by hardware of the system along with a surgical tool that is accommodated by the securing device. The surgical tool is positioned by the securing device at a stable predetermined orientation relative one of a nasal and a temporal eye side location. Once more, the system is configured such that a surgeon may manually direct an implement of the system to control movement of the device and tool, including the ability to pivot a mechanism of the hardware to rotate the device and tool about 180° along one of a vertical axis and a horizontal axis. Therefore, the device and tool may be controllably moved from the one of the nasal and temporal eye side locations to the other of the locations.

### BRIEF DESCRIPTION OF THE DRAWINGS

FIG. 1 is an overview depiction of an operating room employing an embodiment of a surgical support system for eye surgery.

FIG. 2A is a schematic view of the system of FIG. 1 oriented to support a temporal side eye surgery.

FIG. 2B is a schematic view of the system of FIG. 2A rotated about an axis thereof to support a nasal side eye surgery.

FIG. 3A is an enlarged view of an eye taken from FIG. 2A during a temporal side eye surgery.

FIG. 3B is an enlarged view of an eye taken from FIG. 2B during a nasal side eye surgery.

FIG. 4 is an enlarged overview perspective of the system of FIG. 1 during eye surgery.

FIG. 5 is a flow-chart summarizing an embodiment of employing a surgical support system for eye surgery that facilitates stable movement between temporal and nasal side eye procedures.

### DETAILED DESCRIPTION

In the following description, numerous details are set forth to provide an understanding of the present disclosure. However, it will be understood by those skilled in the art that the embodiments described may be practiced without these particular details . Further, numerous variations or modifications may be employed which remain contemplated by the embodiments as specifically described.

Embodiments are described with reference to certain types of vitrectomy probe surgical procedures. In particular, a procedure in which vitreous humor is removed to address vitreous hemorrhage is illustrated. However, tools and techniques detailed herein may be employed in a variety of other manners. For example, embodiments of a vitrectomy probe as detailed herein may be utilized to address retinal detachments, macular pucker, macular holes, vitreous floaters, diabetic retinopathy or a variety of other eye conditions. Regardless, so long as the surgical procedure is aided by the use of a system to facilitate a stable transition from one of a temporal to a nasal side application and vice versa, appreciable benefit may be realized.

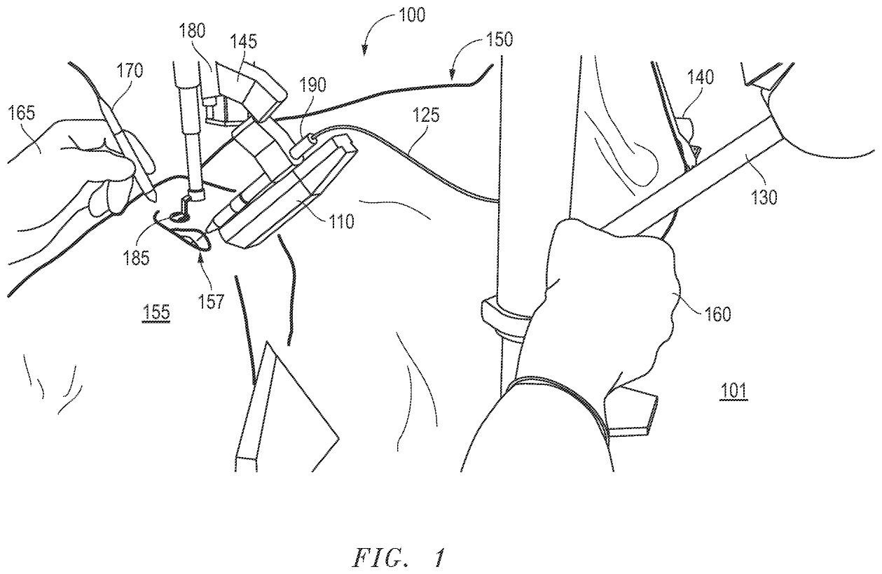

Referring now to FIG. 1, an overview depiction of an operating room 101 is shown employing an embodiment of a surgical support system 100 for eye surgery. The system 100 includes different supportive hardware features such as a device 145 and central 180 supports. Specifically, as described below, the device support 145 may be configured to stably hold a securing device 110 which accommodates a surgical tool 190 during an eye procedure. In the embodiment shown, the device support 145 is coupled to the central support 180 which also serves to accommodate visualization aids for the procedure. For example, a lens guide 185 is illustrated which may be used to directly or indirectly provide enhanced visualization to the surgeon during the procedure.

Continuing with reference to FIG. 1, the system 100 is shown with various components oriented relative a patient 150. Specifically, a patient's head 155 is shown stabilized at one end of an operating table 140 with the patient's eye 157 aligned immediately below the central support 180 and lens guide 185. Thus, a procedure may take place, for example, where the surgeon directs the tool 190, in the form of a vitrectomy probe, to reach into the eye 157 to perform a vitrectomy. In the embodiment shown, the procedure takes place through a pre-placed cannula 225 at the temporal side of the eye 157. More specifically, the procedure is taking place at the patient's right eye 157. Therefore, the temporal side would be an offset location at the right side of the right eye 157 as described further below. Of course, as is also detailed below, the system 100 is also configured to support a procedure through the nasal side as well (i.e., an offset location to the left side of the eye 157).

As with other surgical support systems 100, the system here allows for the surgeon to indirectly control the advancement of the vitrectomy probe needle 190 into and out the eye 157. However, in one embodiment, the movement of the tool 190 relative the eye 157 is limited along a single axis or plane that runs parallel to the securing device 110. That is, as a matter of stability and safety, there may be an intentional limit imposed on the device 110 and tool 190 in terms of movement relative to the eye 157. Regardless, whatever the movement allowed, such may be governed by the surgeon through a motion control toggle 130 (see surgeon hand 160). For example, a pull down on the toggle 130 may translate to a matching and scaled down movement down on the tool 190 into the eye 157. In one embodiment, the toggle movement is scaled down to a 1:7 ratio with every seven degrees of toggle movement translating to a corresponding single degree of tool movement. Of course, other scaled down ratios may be employed which serve to aid the surgeon.

In the embodiment shown, the surgeon is depicted directly manipulating a light instrument 170 (see the surgeon's other hand 165). Note that unlike the tool manipulation, the light instrument 170 is not intended to interact with delicate eye features and may be more suitable for direct handling by the surgeon's hand 165. Of course, in other embodiments, automated light instrument holding could be employed. Additionally, an infusion port may be located at another eye location to maintain volume and pressure at the interior of the eye 157, for example, in light of vitreous humor being removed by the vitrectomy tool 190.

The robotic system 100 may be pneumatic or electric with various connections at the securing device 110. In one embodiment, the device 110 may serve as a universal point of connection for a variety of different surgical eye tools. These may include the illustrated vitrectomy probe 190 which may be pneumatic, laser and/or ultrasound in nature. Once more, a frag or phaco ultrasound tool for cataracts may be utilized, as well as scissors, forceps, picks, shavers, laser probes, a light instrument, a diathermy tool, a spatula, a sweeper, a backflush tool and/or a flex loop tool. The universal drive system with multiple different types of connections at the securing device 110 may be referred to as a draped drive system where connections for electrical wires, pneumatic tubing, vacuum sources and light fibers may be present to support any number of different tool types.

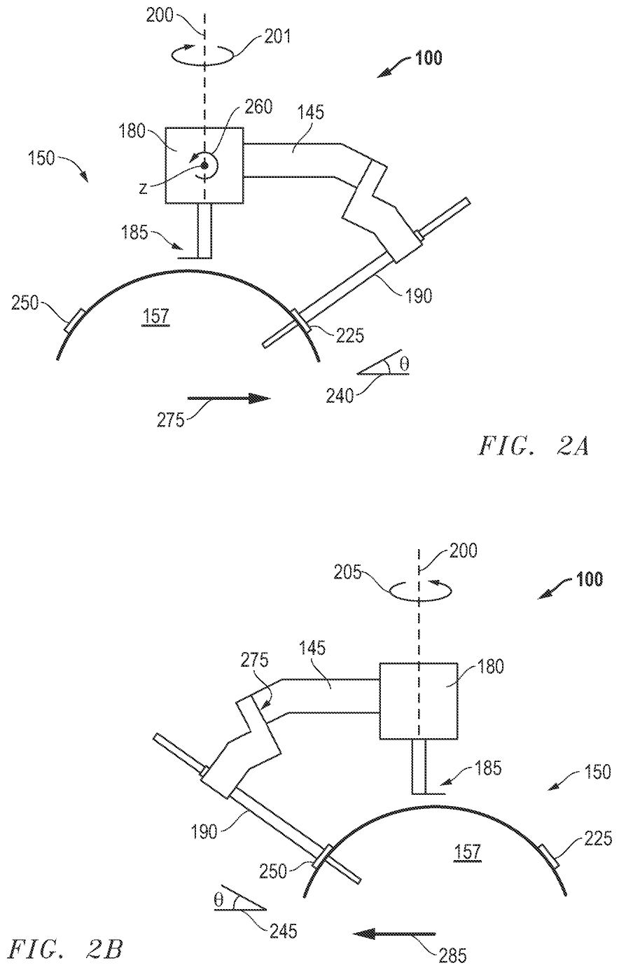

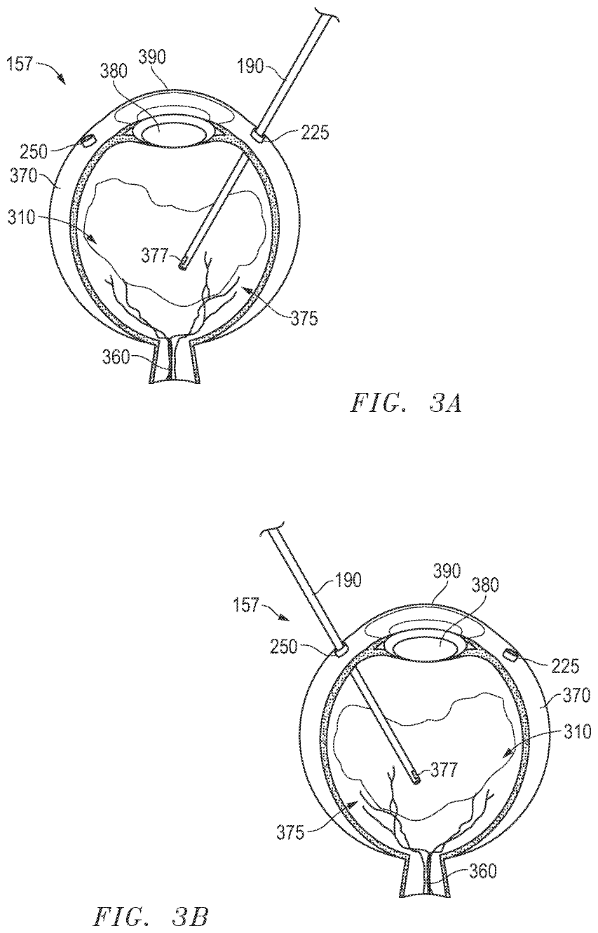

Continuing with reference to FIG. 1, recall that the depicted tool (vitrectomy probe 190), may be limited to movement along a single axis or plane, into or out of the eye interior. Limiting movement in this manner may serve as a safety feature, for example, to prevent other needle movements within the eye 157 from unintentionally harming a side of the eye interior at the underside of the sclera 370, for example (see FIGS. 3A and 3B). This means that the angle between the tool 190 and the eye 157 is fixed. For example, note the angle 240 (θ) of FIG. 2A that is less than about 90°, likely to be between about 30° and 60° with respect to intersecting a temporal side cannula 225.

As detailed below, the system 100 is uniquely configured to allow for a stabilized pivot to another fixed angle 245 of roughly the same degree (θ) as shown at FIG. 2B, at a nasal side cannula 250, when the procedure at the temporal side is complete or otherwise calls for interruption with nasal side intervention. As also detailed below, this pivot 201 may be about 180° about a y-axis 200 as shown in FIGS. 2A and 2B. Alternatively, a pivot may take place about an x-axis to achieve the same reorientation toward a nasal side intervention.

Referring now to FIG. 2A, a schematic view of the system 100 of FIG. 1 is shown oriented to support a temporal side eye surgery. This is a schematic of the same overview illustrated in FIG. 1. However, in the view of FIG. 2A, a vertical y-axis 200 is illustrated about which system hardware (e.g. 180) may be rotated for repositioning of the tool 190. That is, rotation of the central support hardware 180 may take the tool 190 from the temporal side cannula 225 to the nasal side cannula 250 of FIG. 2B as described above.

With specific reference to FIG. 2A, with added reference to FIG. 1, a vitrectomy procedure is run through the temporal side cannula 225 and may take place with the aid of the system 100. The depth of entry into the eye 157 may be precisely guided by the system 100 as the vitrectomy probe 190 traverses the cannula 225. Continued advancement and withdrawal of the probe 190 may be directed by the surgeon with the toggle 130 with the precision assistance of the system 100 and scaling.

In the embodiment shown, the eye 157 is not directly centered below the central hardware 180 for the temporal side application shown but rather slightly offset to the left of center (e.g., offset from the axis 200). However, once the depicted application is complete or suspended for another application at the nasal side cannula 250, the probe 190 may be removed and the patient 150 moved slightly to the right (arrow 275) in relation to the system 100. Of course, this is relative in the sense that the system 100 might instead be moved slightly to the left. With this movement of the patient 150 relative the system 100, a nasal side application may take place.

With added reference to the schematic of FIG. 2B, notice that the system hardware 180, 145 has been rotated (201) about the y-axis 200 such that the device support 145 is now located in alignment with the nasal side cannula 250 for the vitrectomy probe 190. The offset for the nasal side cannula 250 is such that another angular interface 245 is presented for the probe 190. However, this angle 245 roughly matches that of the temporal side cannula 225. For example, in one embodiment, both angles 240, 245 may be about 45°. Of course, any number of other roughly matching acute angle presentations may be utilized. Furthermore, following completion of the depicted vitrectomy procedure at FIG. 2B, there may be a desire to return to an application at the temporal side cannula 225. Thus, the patient 150 may again be slightly moved (see 285) in order to return to temporal side positioning with another rotation 205 of the hardware 180 about the y-axis 200.

The above movement of the system 100 about the axis 200 involves the rotation or pivot of hardware (e.g., 180) about a y-axis. However, in another embodiment, the system 100 may instead rotate 260 about a z-axis point (z) (see FIG. 2A). For example, consider a rotation or pivot 260 about the z-axis point (z) of about 180° from the position shown in FIG. 2A. This would leave the device support hardware 145 in a roughly upside down position. Thus, for this embodiment, a support interface 275 may be rotated so as to re-orient the support hardware 145 into a position for supporting the illustrated nasal side application of FIG. 2B.

Referring now to FIG. 3A, an enlarged perspective view of an eye 157 taken from FIG. 2B is illustrated during a nasal side eye surgery. During the procedure, the probe needle 190 is inserted through a preplaced temporal side cannula 225 and directed toward a region 310 where vitreous humor is to be removed. Specifically, a suction is applied and a port 377 is used for the uptake of the vitreous humor or other substances. For example, in the procedure illustrated, a hemorrhage may be taking place in the region 310 such that blood is drawn into the port 377 along with the vitreous humor.

The surgery includes the probe 190 reaching into the eye 157 through the cannula 225 which is positioned in an offset manner at the sclera 370. In this way, the more delicate cornea 390 and lens 380 may be avoided. This also results in the angular manner of cannula probe interfacing noted hereinabove. The optic nerve 360 and retina 375 are also quite delicate. Therefore, given that the probe needle 190 is capable of reaching these delicate features, utilizing the system 100 of FIG. 1 for precision guidance may be of substantial benefit. Once more, the manner in which this is achieved for the embodiments herein, do not require the surgeon to pause for any eye adjustment for sake of his/her own visibility.

In one embodiment, the system 100 of FIG. 1 may be utilized to not only scale surgeon directives as described above, but also to filter out sudden or unintended movements. For example, a slip or hand tremor of the surgeon may be detectably filtered out by the guidance of the system 100 so as to avoid potentially inducing damage to delicate eye features as noted above. Once more, the degree of precision attributable to the system 100 and scaling may translate to greater than about 25 micrometers. When combined with the safe efficiency of translating the system 100 from temporal to nasal side cannula interfacing and/or vice versa, the degree of safety and efficiency may be substantially enhanced through use of the system 100.

Referring now to FIG. 3B, an enlarged view of an eye 157 taken from FIG. 2B during a nasal side eye surgery is illustrated. As described above, a simple rotation, likely about 180°, may be utilized to reposition the system and surgical tool 190 into appropriate orientation for reaching through the nasal side cannula 250. In one embodiment, even though the range of motion for the tool 190 may be safely limited to a linear ingress and egress relative the interior of the eye 157, the ability to efficiently move between cannulas 125, 150 with the indicated angular interfacing, means that the majority of the eye interior is nevertheless reachable by the tool 190. Thus, efficiency and safety are both enhanced without substantial drawback to the imposing of a limited range of motion on the tool 190.

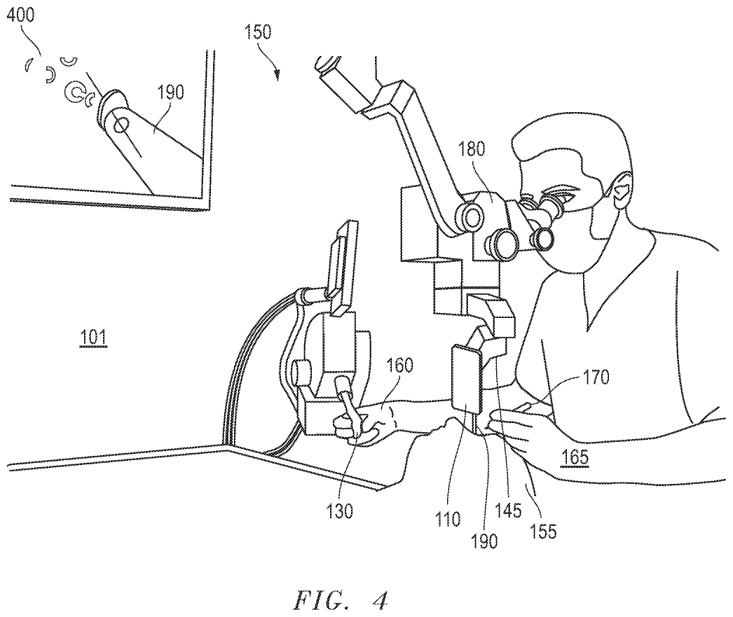

Referring now to FIG. 4, an enlarged overview perspective of the system 100 of FIG. 1 is illustrated during eye surgery. In this view, the surgeon is shown manipulating the surgical tool 190 by way of the toggle 130 at the surgeon's right hand 160. At the same time a light instrument 170 is directly manipulated by the surgeon with a left hand 165. While the surgeon's view is through a visual aid associated with the central hardware 180, another manner of visualization is available to the surgeon or others in the form of a larger mounted video screen 400 (e.g., depicting the surgery and vitrectomy tool 190 within the eye).

Utilizing such a system may afford an improved degree of precision, perhaps better than 30 micrometers with the scaling described above. Further, in one embodiment, the securing device 110 may be outfitted with a host of devices, for example, in addition to the described vitrectomy probe. That is, rather than exchanging surgical tools with each application at a given side of the eye, the securing device 110 may be of a turret or carousel configuration accommodating multiple tools at the same time. In the embodiment shown, the securing device 110 is oriented to driving an application through a vitrectomy probe 190. However, a tool supply carousel rotated to provide forceps or scissors or any other instrument, may be utilized to facilitate the securing device engagement therewith. Thus, even before pivoting to another side of the patient's eye or even to another eye, a surgical tool change out may take place with further applications directed at the same eye side location.

The ability of the securing device 110 to switch out devices automatically may involve the use of patient specific disposable devices. Further, as suggested above, the securing device 110 would likely include multiple connection types to support varying device types. Thus, the securing device 110 and couplings may constitute a universal drive source.

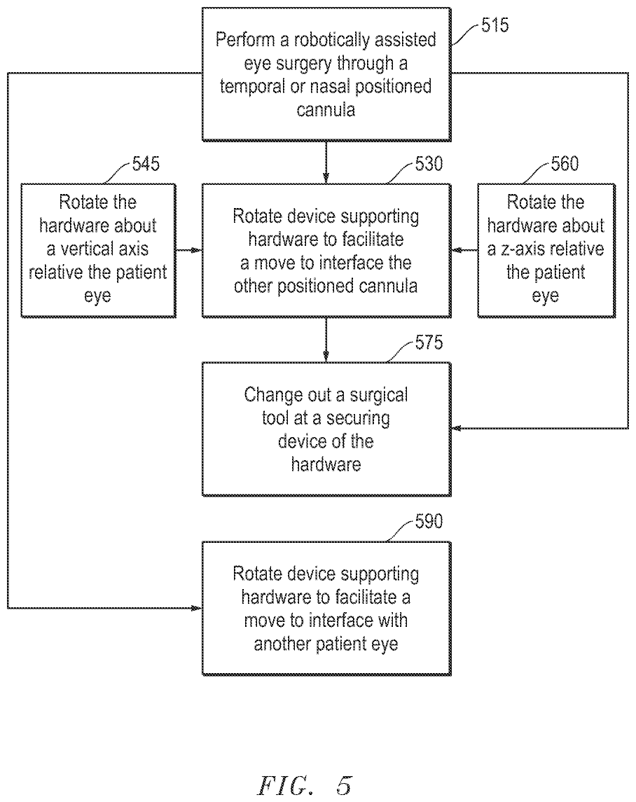

Referring now to FIG. 5, a flow-chart summarizing an embodiment of employing a surgical support system for eye surgery is illustrated that facilitates stable movement between temporal and nasal side eye procedures. Namely, a robotically assisted eye procedure may be guided by the system through one of the temporal and nasal side (see 515). This may be followed by rotating the device supporting hardware of the system to the other of the locations as indicated at 530. This may occur by rotation of the hardware about a vertical axis relative the patient (see 545) or by rotation about a z-axis relative the patient (see 560). Once more, as noted at 575, the movement between eye side locations may be accompanied by any number of device changeouts. Indeed, in one embodiment, the rotation may be one from one eye to another and not just between sides of the same eye (see 590).

Embodiments described hereinabove include a robotically assisted eye procedure that enhances efficiency when a surgical entry site moves from one eye side to another and/or between eyes. Specifically, in spite of a safety-based, limited range of motion for the system, hardware thereof may be configured to rotate about an axis from one stabilized position at one eye side to another stabilized position at another eye side or even to another eye. As a result, the need to undertake significant repositioning of the system or even the patient may be eliminated without the need to forego limited range of motion safety measures for the system and patient. Thus, eye surgery efficiencies may be dramatically enhanced.

The preceding description has been presented with reference to presently preferred embodiments. However, other embodiments and/or features of the embodiments disclosed but not detailed hereinabove may be employed. Furthermore, persons skilled in the art and technology to which these embodiments pertain will appreciate that still other alterations and changes in the described structures and methods of operation may be practiced without meaningfully departing from the principle and scope of these embodiments. Additionally, the foregoing description should not be read as pertaining only to the precise structures described and shown in the accompanying drawings, but rather should be read as consistent with and as support for the following claims, which are to have their fullest and fairest scope.

1. An automated surgical support system to facilitate eye surgery, the system comprising:
a tool securing device supported by hardware of the system;
a surgical tool accommodated by the device at a stable predetermined orientation relative one of a nasal and a temporal eye side location;
a surgeon directed manipulatable toggle to control movement of the device and tool; and
a pivot mechanism of the hardware configured to rotate the device and tool about 180° along one of a vertical axis and a horizontal axis form the one of the nasal and temporal eye side locations to the other of the locations.

2. The surgical system of claim 1 wherein the predetermined orientation is of an angle that is less than about 90°.

3. The surgical system of claim 2 wherein the angle is between about 30° and about 60°.

4. The surgical system of claim 3 wherein the angle is at about 45°.

5. The surgical system of claim 1 wherein the stable predetermined orientation limits a range of movement for the tool to a single plane of movement.

6. The surgical system of claim 5 wherein the single plane of movement is further limited to a single axis of ingress and egress for the tool relative the eye.

7. An automated surgical support system to facilitate eye surgery, the system comprising:
a tool securing device supported by hardware of the system;
a surgical tool accommodated by the device at a stable predetermined orientation relative one of a nasal and a temporal eye side location;
a surgeon directed manipulatable toggle to control movement of the device and tool; and
a pivot mechanism of the hardware configured to rotate the device and tool along one of a vertical axis and a horizontal axis form the one of the nasal and temporal eye side locations to the other of the locations.

8. The automated surgical support system of claim 7 further comprising one of a light instrument and an infusion port to interface with another location of the eye.

9. The automated surgical support system of claim 7 wherein the tool is selected from a group consisting of a vitrectomy probe, a frag ultrasound tool, a phaco ultrasound tool, scissors, forceps, a pick, a shaver, a laser probe, a light instrument, a diathermy tool, a spatula, a sweeper, a backflush tool and a flex loop tool.

10. The automated surgical support system of claim 7 wherein the tool securing device is coupled to a universal draped drive with multiple tool connections.

11. The automated surgical support system of claim 10 wherein the tool securing device is coupled to one of a turret and a carousel accommodating a plurality of different surgical tools.

12. A method of performing a robotically assisted eye surgery, the method comprising:
performing a surgical procedure with a surgical tool accommodated by a surgical system, the tool positioned through one of a temporal and a nasal side positioned cannula at an eye of a patient;
utilizing a surgeon directed manipulatable toggle of the system to control movement of the tool during the performing; and
pivoting hardware of the system to reposition the tool through the other of the temporal and nasal side positioned cannulas.

13. The method of claim 12 wherein the pivoting of the hardware is about one of a vertical axis and a horizontal axis relative the patient.

14. The method of claim 12 further comprising pivoting hardware of the system to reposition the tool to another eye of the patient.

15. The method of claim 12 further comprising:
employing one of a turret and a carousel accommodating a plurality of additional surgical tools to exchange the surgical tool for an additional surgical tool; and
performing further eye surgery with assistance of the system with the additional surgical tool.
